(19) United States
(12) Patent Application Publication
(10) Pub. No.: US 2017/0106149 A1
(43) Pub. Date: Apr. 20, 2017

(54) Single-Use, Self Destructing Shield for Syringe Needle

(71) Applicant: MedImmune, LLC, Gaithersburg, ME (US)

(72) Inventor: Corbin Clawson, Gaithersburg, MD (US)

(21) Appl. No.: 15/128,118

(22) PCT Filed: Mar. 23, 2015
(86) PCT No.: PCT/US15/21942

§ 371 Date: Sep. 22, 2016

Related U.S. Application Data
(60) Provisional application No. 61/969,462, filed on Mar. 24, 2014.

(51) Int. Cl. A61M 5/32; A61M 5/50

(52) U.S. Cl. CPC A61M 5/3202; A61M 5/3213; A61M 5/5086

(57) ABSTRACT

Disclosed are embodiments of a cap covering a needle on a syringe. The cap in the closed configuration is sized and dimensioned to cover the needle, and after the cap is removed from the needle the cap assumes an open configuration that is not readily usable to re-cap the needle.

This application claims priority to U.S. provisional patent application having Ser. No. 61/969,462, filed Mar. 24, 2014, the contents of which are incorporated herein by reference in their entirety.

### FIELD OF THE INVENTION

This invention generally relates to a single-use shield or cap for a syringe needle and the like, which after the first and only use the shield or cap assumes a shape that discourages recapping of the exposed needle.

### BACKGROUND OF THE INVENTION

A significant number of needle stick injuries, i.e., about 30%, occurs during disposal of used syringes and needles. A large portion of those injuries happen when the users attempt to re-cap the needles after they have been used. Despite industry guidance against recapping used needles, many users still perceive a recapped needle to be safer than an exposed needle, and needle stick injuries remain high. Standard needle caps, or rigid needle shields, as they are known in the art, do not communicate to the user through their form or design that they should not be recapped. In fact, it could be argued that they communicate the opposite, as their form resembles that of pen caps or other items that users typically recap.

There are some cap designs that prevent the cap from being securely replaced and would simply fall off if the user tries to recap it. This design, however, requires the user to learn that the cap cannot be replaced by first attempting to replace the cap and then seeing that it would not securely cap. These attempted recaps can result in needle stick injuries, and the users can still be injured with this design.

There remains a need for cap designs that readily communicate to the user that the caps after being removed from the needle or syringe should not be used to recap the exposed needle.

### SUMMARY OF THE INVENTION

Hence, the invention is directed to needle caps that once removed from covering the needle communicate to the user preferably through the form, function and/or appearance of the caps that the caps are not meant to be replaced or repositioned to cover the needle.

The invention is also directed to needle caps that once removed from the needle substantially change their shape permanently or self-destruct to communicate to the users that the caps do not go back onto the needle.

### BRIEF DESCRIPTION OF THE DRAWINGS

In the accompanying drawings, which form a part of the specification and are to be read in conjunction therewith and in which like reference numerals are used to indicate like parts in the various views:

FIG. 1 is a top view of a syringe with a conventional needle cap;

FIG. 2(a) is a top view of an embodiment of the inventive needle cap in the before use or first configuration; FIG. 2(b) is a top view of the inventive needle cap in the after use or second configuration; and FIG. 2(c) is an exploded cross-sectional view of a live hinge joint;

FIGS. 3(a)-(c) are other embodiments of the inventive cap;

FIG. 4(a) is a top view of another embodiment of the inventive needle cap in the before use or first configuration; FIG. 4(b) is an end view of the of the needle cap of FIG. 4(a); and FIGS. 4(c) and 4(d) are end views of the inventive needle cap of FIGS. 4(a) and 4(b) in the after use or second configuration; and

FIG. 5 is a schematic illustration of the embodiment shown in FIGS. 4(a)-4(d).

### DETAILED DESCRIPTION OF THE PREFERRED EMBODIMENTS

Needle shields/caps are used with syringes. Syringes consist of a hollow barrel fitted with a plunger and, may be cartridges, hypodermics, single use syringes, multiple use syringes, auto-injectors, and the like. In order to communicate to the user that needle shields/caps onced used and removed from the syringe should not be used to recap the exposed needles, the present invention provides a self-destructing needle cap or a needle cap that substantially permanently changes its shape or configuration after the first use. The inventive needle cap in one embodiment consists of two or more segments hinged at the top, but free to extend or expand like flower petals. When placed over the needle, the petals would close around the needle and be secured by a retaining structure. The petals may be secured by a variety of structures, including but not limited to, a slideable band over the petals and an overhanging feature from the syringe to retain the petals, or be attached by breakable connections to a stationary ring attached to the syringe, much like a bottle cap connected to a lower ring for tamper detection. The petals may also be secured by a shrink wrap plastic sleeve that has a scored or weakened element or that has an embedded string to tear the wrap, similar to the safety shrink wrap sleeves common to over-the-counter medicines. Once the user removes the cap from the needle, the petals or segments would then expand and open like a flower opening its petals.

The petals could be sprung open by pressure from an elastomer core, which is a common needle shield for prefilled syringes that incorporates a rigid plastic shield with an elastomer core inside to seal the needle. The core is compressed when the petals are secured together, or from a geometric feature of the rigid cap itself molded into the plastic. Suitable geometric features include, but are not limited to, a shape memory alloy (SMA) that remembers its shape and connects the petals to the cap. In another embodiment, the petals form a live hinge with the remaining portion of the cap and when secured to the other petal(s) form a spring joint or a live hinge joint, discussed below. Thus the needle cap, in a sense, self-destructs and it is clear to the user that it would no longer function as a needle cap. The user would intuitively know that the cap could not and should not be replaced, and so would not attempt to replace it, thus eliminating the risk of needle stick injury. Another benefit of the self-destructing needle shield is that it can also serve as a tamper evident closure and anticounterfeiting protection.

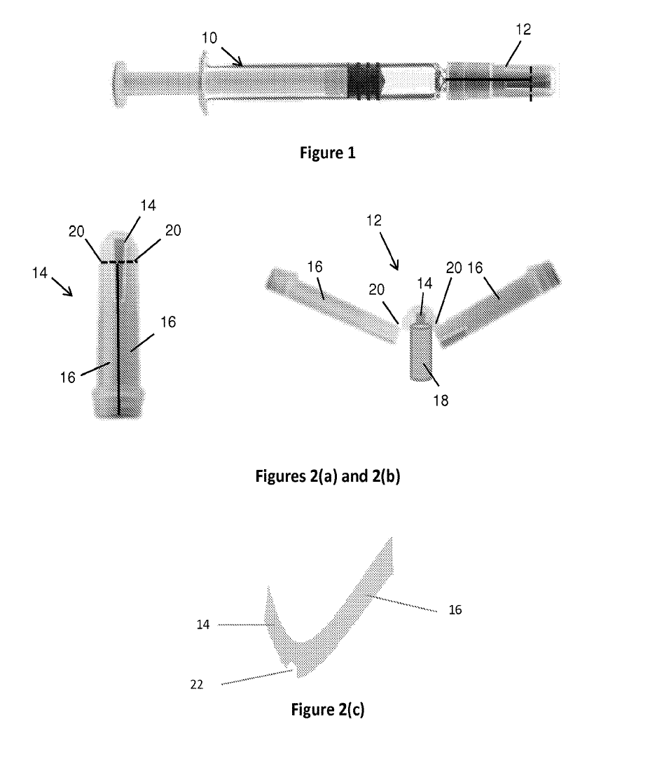

Referring to FIG. 1, syringe 10 with an inventive needle cap 12 is shown. Cap 12 covers a hypodermic needle that injects a medicine or vaccine or the like into a patient. A first embodiment of inventive needle cap 12 is shown in FIGS. 2(a) and 2(b). Cap 12 comprises tip 14 with petals 16. While two petals 16 are illustrated in this embodiment, a single or two or more petals 16 can be employed and the present invention is not limited to any particular number of petals. Within cap 12, preferably elastomeric core 18 is present and a needle 19 is pushed into and sealed by core 18 (see FIG. 5).

Cap 12 can be molded from a polymeric plastic, and as molded has the open configuration or shape shown in FIG. 2(b), i.e., with the petals opened. Petals 16 are joined to tip 14 by at least one live hinge 20. A live hinge is a flexible hinge made from the same material as the two pieces it connects. The live hinge is generally thinner than the two connecting pieces or is cut to allow two more rigid connecting pieces to bend along the line of the live hinge. Live hinge 20 is therefore thinner or has a smaller thickness than the walls of tip 14 and petals 16 that it joins, as best shown in FIG. 2(c). Notch 22 of hinge 20 allows petals 16 to rotate downward toward each other for the cap 12 to be assembled and placed on the needle, as shown in FIGS. 1 and 2(a). Notch 22 may also be positioned on the inside surface as shown or on the outside surface. The low cost and ease of manufacturing makes live hinges suitable for disposable packaging. Plastic living hinges are typically manufactured in an injection molding operation or in a compression molding that creates all three parts, i.e., tip 14, petals 18 and hinge 20, at one time as a single integral part. Suitable plastic materials include, but are not limited to, polyethylene and polypropylene. Hinge 20, tip 14 and petals 16 can also be compression molded from thermosetting or thermoplastic elastomeric materials. Core 20 is preferably made separately, or alternatively may be molded at the same time as the rest of cap 12 using known co-injection technique and can be made with a material that is different than the materials for the rest of the cap.

Preferably, cap 12 is molded in the open shape shown in FIG. 2(b) and this open shape is at least partially set when molded, so that cap 12 remembers this open shape and at least partially returns to this open shape when a force or a retainer holding the petals together is removed.

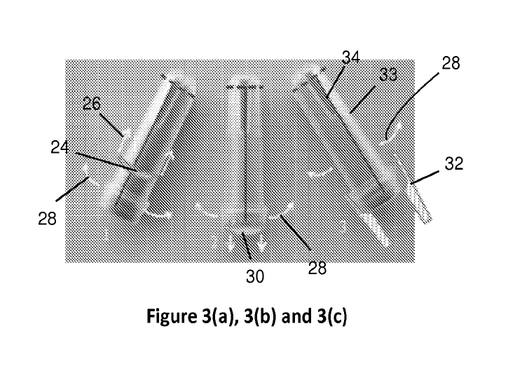

Petals 16 are secured together to the closed configuration or shape of FIG. 2(a) by a variety of means shown in FIGS. 3(a)-3(c). A band 24, which can be flexible or rigid, can be slid over petals 16 as shown in FIG. 3(a) to secure petals 16 to each other. As petals 16 are pressed toward each other, they may compress elastomeric core 18. To remove cap 12 from the needle, band 24 is moved along direction 26 toward tip 14. After band 24 is removed, live hinges 20 and the shape memory of molded open cap 12 push petals 16 apart in direction 28. The compression of elastomeric core 18 provides an optional force that pushes petals 16 apart, when band 24 is removed.

Cap 12 can also be attached via a number of frangible bridges to a lower ring 30, which is attached to syringe 10, as shown in FIG. 2(b). Ring 30 is similar to the lower ring on a tamper evident seal that is connected to caps to soda bottles and condiment bottles, among others. When lower ring 30 is removed from cap 12, for example, by a twisting motion to break the frangible bridges, in direction 30, petals 16 open in direction 28. Advantageously, the compression of elastomeric core 18 when released provides the force that pushes petals 16 apart.

In another embodiment, cap 12 is held in the closed configuration of FIG. 2(a) by a number of overhanging arms 32 from syringe 10, shown in FIG. 3(c). Arms 32 secure petals 16 together. In one variation, the distal ends of arms 32 are flexible, so that cap 12 can be pulled away from syringe 10 in direction 26 and petals 16 open in direction 28. In another variation, arms 32 are pivotable, and when the proximal ends of arms 32 are pressed together the distal ends move away from each other to release cap 12 and petals 16.

In another embodiment, cap 12 is kept in the closed configuration by wrap 33, preferably a heat shrink wrap. Such a wrap may have scored line(s) such as the line designated by reference number 34. Alternatively, the wrap may have a thread or the like which can also be designated by reference number 34. The user may break or tear the wrap at scored line 34 or tear the thread 34 to release petals 16. Advantageously, wrap 33 may extend over a portion of the barrel in syringe 10, such that the user needs to break or tear wrap 33 in order to remove cap 12 from syringe 10. The same motion that tears wrap 33 to allow cap 12 to be removed also releases petals 16.

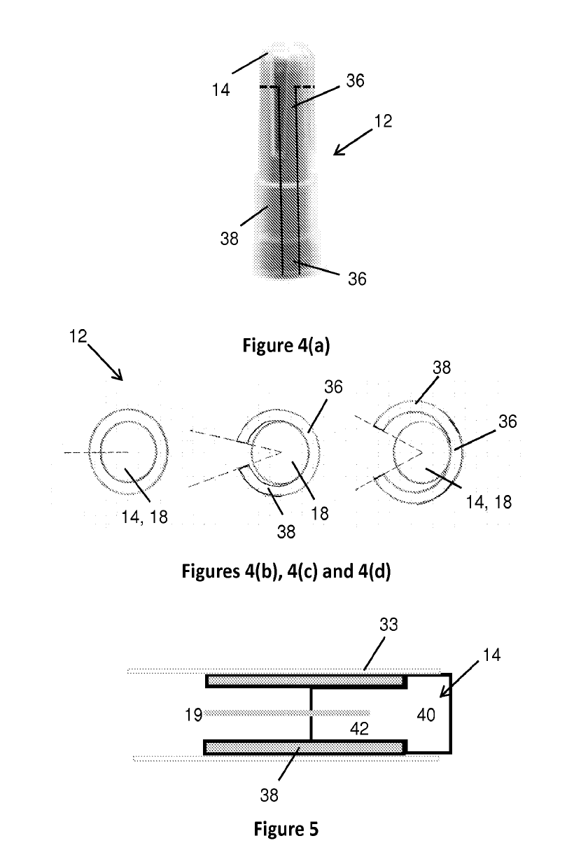

Yet another embodiment is illustrated in FIGS. 4(a)-4(d). Live hinge 36 is located along the longitudinal axis of cap 12, as best shown in FIG. 4(a), and only one petal 38 is necessary for each live hinge 36. For simplicity, one live hinge 36 and one petal 38 is used and illustrated. Live hinge 36 may also have a notch running along the longitudinal direction either on the inside surface or the outside surface. Cap 12 can also be molded in the open shape shown in either FIG. 4(c) or 4(d), and petal 38 is pressed together to from the closed configuration shown in FIG. 4(b). The larger opening in petal 38, e.g. shown in FIG. 38, provides a higher shape memory and therefore a higher opening force. An advantage of this embodiment is that live hinge 36 is larger and can provide more opening force. Additionally, the molded open shape is simpler to manufacture.

In another embodiment, to further simplify the molding/manufacturing process tip 14 may be omitted and petal 38 can be extruded and then cut to any desired length, as illustrated in FIG. 5. This obviates the need to mold parts with complicated geometries. Elastomeric core 18, which secures the tip of needle 19 of the syringe, can have a section 40 with a larger diameter and a section 42 with a smaller diameter. Petal 38 without tip 14 can be wrapped around the smaller section 42 so that the outside surface of petal 38 is flushed with the larger section 40 to give cap 12 a smooth outer surface. Wrap 33 discussed above can be used to keep cap 12 in the closed configuration of FIG. 4(b). Preferably, wrap 33 also covers at least a portion of syringe 10 or all of syringe 10.

Alternatively, preferably for syringes that are not pre-filled core 18 may be omitted as long as petal 38 in the closed configuration and acting as a sheath extends safety beyond the distal end of needle 19 to prevent access to needle 19. Wrap 33 may cover the open end of petal 38 to maintain sterility and cleanliness to needle 19.

Live hinges are optional in the embodiment of FIGS. 4(a)-(c) and of FIG. 5, because the single petal 38 itself acts as a spring when compressed to form the closed configuration. Live hinges, including notch 22, can be included to control, e.g., lower, the spring force of petal 38.

While it is apparent that the illustrative embodiments of the invention disclosed herein fulfill the objectives stated above, it is appreciated that numerous modifications and other embodiments may be devised by those skilled in the art. Feature(s) from one embodiment can be incorporated into other embodiments. Therefore, it will be understood that the appended claims are intended to cover all such modifications and embodiments, which would come within the spirit and scope of the present invention.

What is claimed is:

1. A cap in a closed configuration sized and dimensioned to cover a needle of a syringe comprising:
at least one petal retained in the closed configuration while covering the needle by a retaining member, wherein when the retaining member is removed a force stored within the cap opens the at least one petal to an open configuration, wherein in the open configuration the cap is not sized and dimensioned to cover the needle.

2. The cap of claim 1 further comprising a core attached to the distal end of the needle in the first configuration.

3. The cap of claim 2, wherein the core is elastomeric and in the closed configuration is compressed and in the open configuration provides said force to open the at least one petal.

4. The cap of claim 1, wherein the cap possesses a shape memory associated with the open configuration that provides said force to open the at least one petal.

5. The cap of claim 1 further comprising a live hinge connecting the at least one petal to the remainder of the cap.

6. The cap of claim 5 further comprising a tip wherein the live hinge connects the at least one petal to the tip.

7. The cap of claim 5, wherein a notch is provided proximate to the live hinge.

8. The cap of claim 6, wherein at least two petals are connected by the live hinge and in the open configuration the at least two petals rotate about the live hinge to open.

9. The cap of claim 1, wherein the retaining member comprises a ring fitted over the petal in the closed configuration.

10. The cap of claim 1, wherein the retaining member comprises a lower ring attached to the cap by frangible bridges.

11. The cap of claim 1, wherein the retaining member comprises a plurality of elongated arms extending from the syringe.

12. The cap of claim 1, wherein the retaining member comprises a wrap.

13. The cap of claim 1, wherein the wrap comprises a heat shrink wrap.

14. The cap of claim 13, wherein the wrap comprises at least one scored line or at least one embedded string for tearing the wrap.

15. The cap of claim 12, wherein the cap covers at least a portion of the syringe.

16. A method for unsheathing a needle comprising the steps of
(a) providing a cap having a closed configuration and an open configuration
(b) covering the needle with said cap in the closed configuration;
(c) removing a retainer that keeps the cap in the closed configuration;
(d) allowing the cap to move the open configuration, wherein the closed configuration is not sized and dimensioned to cover the needle.
